(19) United States
(12) Patent Application Publication
(10) Pub. No.: US 2015/0366585 A1
(43) Pub. Date: Dec. 24, 2015

(54) TENSION-LIMITING TEMPORARY EPICARDIAL PACING WIRE EXTRACTION DEVICE

(71) Applicants: Matthieu Olivier Lemay, Kingston (CA); Fraser Douglas Rubens, Ottawa (CA)

(72) Inventors: Matthieu Olivier Lemay, Kingston (CA); Fraser Douglas Rubens, Ottawa (CA)

(21) Appl. No.: 14/743,128

(22) Filed: Jun. 18, 2015

Related U.S. Application Data
(60) Provisional application No. 62/013,825, filed on Jun. 18, 2014.

(51) Int. Cl. A61B 17/50; B65H 54/00; B65H 59/40; B65H 59/00

(52) U.S. Cl. CPC A61B 17/50; B65H 59/00; B65H 54/00; B65H 59/40; A61B 2017/00128

(57) ABSTRACT

A tension-limiting temporary epicardial pacing wire extraction system provides an alternative to current temporary epicardial pacing wire extraction methods in order to reduce the risk of severe complications that may result from variable and excessive tension being applied to the wire during manual extraction. The epicardial pacing wire extraction system includes a housing that houses a motor, a handle for holding the device, a cartridge containing a spool driven by the motor through a coupling for extracting an epicardial pacing wire from a patient, a start/stop button to operate the motor to drive the spool and a cartridge-release mechanism that selectively releases the cartridge from the housing.

### CROSS-REFERENCE TO RELATED APPLICATIONS

This application claims the benefit of priority of U.S. Provisional Application Ser. No. 62/013,825, filed on Jun. 18, 2014, the benefit of priority of which is claimed hereby, and which is incorporated by reference herein in its entirety.

### TECHNICAL FIELD

The present invention relates generally to epicardial pacing wires and, in particular, to methods and devices for extracting such wires.

### BACKGROUND

Temporary epicardial pacing wires (TEPW) are commonly used during the postoperative care of patients who have undergone cardiac surgery. As illustrated in FIG. 1, a TEPW generally consists of a thin insulated metal wire (2), one end of which has a small electrode (1) while the other end has a breakaway needle (5). During cardiac surgery, the electrode is implanted into the surface of the heart by passing the curved needle (4) through the heart tissue. As the electrode passes through the tissue behind the curved needle, the coiled plastic filament (3) provides enough resistance to fix the electrode to the tissue. Once the electrode is secure, the curved needle is cut off from the end of the wire. Next, the breakaway needle is passed through the abdomen wall from the inside, just below the ribs. Its sharp tip is broken off leaving behind a portion of the needle shaft which is used to connect the wire to an external pacemaker. In the event of temporary postoperative problems with the native rhythm, such as a slow heart rate or bradycardia, electrical pulses can be sent from the pacemaker, through the wire, to the electrode in the heart tissue, causing the heart muscles to contract.

In most institutions, TEPW removal is a “designated medical act” whereby it is performed by nursing staff under the responsibility of an attending cardiac surgeon. Generally, the TEPW is removed from the patient within a few days of the surgical procedure when it is deemed that it is no longer needed. The current extraction method involves grasping the external portion of the TEPW by hand and gently pulling until the electrode dislodges from the heart tissue and the wire is completely removed through the small puncture hole in the abdomen.

Teaching TEPW removal is extremely difficult due to the subjective nature of describing the degree of tension needed to safely extract the wire. In addition, there are currently no explicit guidelines to govern the maximum amount of tension to be used. As a result, the application of traction is left to the interpretation of the healthcare professional performing the procedure. Although most TEPW removals are performed without any problem, in some instances very serious and life threatening complications can occur, including entrapment of the wire and cardiac tamponade. In certain cases, these complications can be attributed to excessive and variable use of traction force when pulling on the TEPW. In the event that the nurse performing the procedure has deemed the tension to be excessive, he or she is instructed to abandon the procedure and cut the wire flush with the skin. Leaving the bulk of the wire attached to the heart has been shown to precipitate late infection as a foreign body, chronic pain, and other complications. Furthermore, patients with retained TEPW may not be eligible to undergo magnetic resonance imaging with 3 Tesla magnets, thus potentially preventing these patients from undergoing important procedures such as neurologic exams. In summary, the problem with the current method of TEPW removal lies in the uncertainty in the maximum traction to be applied to the wire without compromising the wellbeing of the patient.

U.S. Patent Application Publication 2009/0234367 (Verma) discloses a force assessment device and method for extraction of endocardial pacing leads (i.e. leads implanted inside the heart). The device includes a force gauge and a strain gauge for measuring traction and counter-traction forces and a display for presenting force readings, information or alerts to the user. The device includes a release mechanism to release a stylet from a handle of the device when a traction force exceeds a predefined threshold. The device is also configured to telescopically contract a sheath when a counter-traction force exceeds a predefined threshold. This device provides a tool for extracting endocardial pacing leads, however, the Applicant is unaware of any device designed specifically for removing epicardial pacer wires (i.e. leads implanted on the surface of the heart). As such, it would be highly desirable to provide an ergonomic device for safely removing epicardial pacer wires, specifically. It would also be highly desirable to provide a means for capturing or containing the pacer wire after its extraction.

### SUMMARY

In response to the foregoing problem, the tension-limiting temporary epicardial pacing wire extraction device (henceforth referred to as the “device”) was developed with the intention of providing an alternative to current TEPW extraction methods. By automating the extraction process while closely monitoring the tension applied to the TEPW, the device may reduce the element of uncertainty and the risk of severe and life-threatening complications associated with the procedure. The device is meant to be used predominantly by nursing staff although it may also be utilized by a physician, medical school, resident or any other technician or worker, depending on the jurisdiction and local practice.

The device's primary function is to apply continuous tension to the extracorporeal portion of the TEPW in order to dislodge the electrode from the heart tissue and completely remove the wire from the patient's body. A disposable cartridge, designed to be used along with the device, contains a spool to which the extracorporeal end of the TEPW is attached. The device is placed on the patient's abdomen in proximity to where the wire passes through the skin and the loaded cartridge is attached to the device. Attaching the cartridge connects the spool with the device's powertrain. As a result, when the device's trigger is pressed, the torque produced by the device is transferred to the spool, which in turn pulls on the TEPW as it is wound around the spool.

At the same time, the device continuously monitors the applied tension on the wire. If the tension rises and approaches a predetermined maximum force threshold, audible, visible, and tactile feedback is given to warn the operator. In the case that the tension force reaches the maximum threshold, the device will release the cartridge, immediately eliminating any tension on the wire. These thresholds can be programmed into the device by the operator and may vary depending on the location of the TEPW's electrode on the heart. Recent tests performed by one of the inventors suggest that the tension to be applied on the wire should not at any time exceed 44 oz. In addition, the device includes safety features that protect the patient from injury in the event that the device is misused, dropped, or otherwise not performing as intended.

When the TEPW has been completely extracted and wound around the spool, the device is removed from the patient. The cartridge is released and discarded in compliance with hospital protocol.

Accordingly, an inventive aspect of the disclosure is an epicardial pacing wire extraction system that includes an epicardial pacing wire extraction device having a housing that houses a motor, a handle for holding the device, a start/stop button to operate the motor, and a cartridge-release mechanism. The system also includes a cartridge selectively releasable by the cartridge-release mechanism. The cartridge contains a spool driven by the motor for extracting an epicardial pacing wire from a patient. The cartridge may be designed to lock on closure to prevent its reuse.

Another inventive aspect of the disclosure is an epicardial pacing wire extraction device comprising a housing that houses a motor, a handle for holding the device, a start/stop button to operate the motor and a cartridge-release mechanism.

Yet another inventive aspect of the disclosure is a cartridge for use with an epicardial pacing wire extraction device to extract an epicardial pacing wire. The cartridge includes a casing for containing the epicardial pacing wire during and after extraction and a spool held inside the casing, the spool attaching to an external end of the epicardial pacing wire, the spool having an external portion driven by a torque-transmitting coupling of the device. The cartridge may be designed to lock on closure to prevent its reuse.

This summary is intended to highlight certain significant inventive aspects but is not intended to be an exhaustive or limiting definition of all inventive aspects of the disclosure. Other inventive aspects may be disclosed in the detailed description and drawings.

### BRIEF DESCRIPTION OF THE DRAWINGS

Further features and advantages of the present technology will become apparent from the following detailed description, taken in combination with the appended drawings, in which:

FIG. 1 depicts a temporary epicardial pacing lead as known in the prior art;

FIG. 2 depicts an operational block diagram of the tension-limiting temporary epicardial pacing wire extraction device;

FIG. 3A is an exploded view of a cartridge for use with an epicardial pacing wire extraction device;

FIG. 3B is an exploded view of the cartridge;

FIG. 3C is an isometric view of a first version of the spool;

FIG. 3D is an isometric view of a second version of the spool;

FIG. 3E is an isometric view of the cartridge;

FIG. 3F is an isometric view of the cartridge;

FIG. 4 is an isometric view of the device;

FIG. 5A is an isometric cutaway view of the cartridge holder and release mechanism in a first configuration;

FIG. 5B is an isometric cutaway view of the cartridge holder and release mechanism in a second configuration;

FIG. 5C is an isometric cutaway view of the cartridge holder and release mechanism in a third configuration;

FIG. 5D is an isometric cutaway view of the cartridge holder and release mechanism in a fourth configuration;

FIG. 6 is an exploded view of the powertrain;

FIG. 7A is a side cutaway view of a wire extraction mechanism, tension measurement mechanism, and cartridge release mechanism;

FIG. 7B is an enlarged side cutaway view of the wire extraction mechanism, tension measurement mechanism, and cartridge release mechanism;

FIG. 8 an isometric cutaway view of the device in accordance with one embodiment of the invention.

It will be noted that throughout the appended drawings, like features are identified by like reference numerals.

### DETAILED DESCRIPTION

Disclosed herein are one or more embodiments of an epicardial pacing wire extraction system. In general, the system includes an extraction device and a replaceable (disposable) cartridge. The device includes a housing that houses a motor, a handle for holding the device. The cartridge contains a spool driven by the motor for extracting an epicardial pacing wire from a patient. The device includes a start/stop button to operate the motor to drive the spool, and a cartridge-release mechanism to selectively release the cartridge from the device.

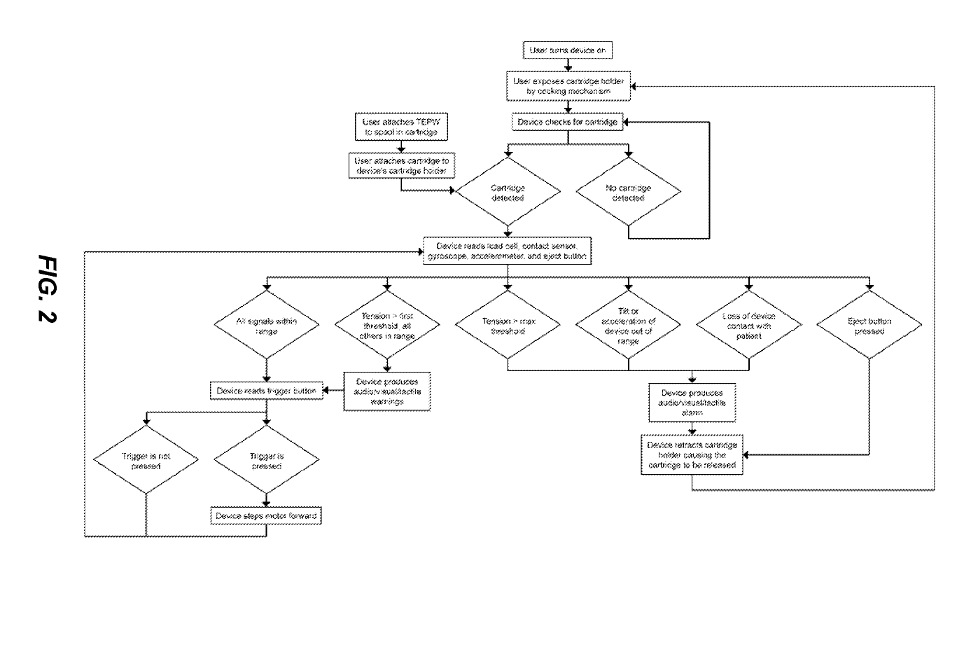

FIG. 2 depicts an operational block diagram for the device. As depicted in FIG. 2, after the device is turned on, the user exposes a cartridge holder by pushing forward on the slider (208). As shown in FIG. 2, the user attaches the TEPW to the spool in the cartridge and attaches the cartridge to the cartridge holder. The device (or more specifically a microprocessor, microcontroller, control circuit or other control logic in the device) is configured to check for a cartridge. In response to detecting the presence of a cartridge, the device reads one or more signals from a load cell, contact sensor, gyroscope, accelerometer and eject button. If all signals are within the desired respective ranges, the device becomes operable and responds to trigger button input from the user to step the motor forward. If the tension is above a first threshold (but all other signals are within range), the device outputs a warning, e.g. an audible, visual and/or tactile warning. If the tension exceeds a maximum threshold, the device outputs a warning and releases the cartridge as a safety measure. Likewise, the device outputs a warning and releases the cartridge as a safety measure if the tilt or acceleration is out of range, or if the devices loses contact with the patient, or if the eject button is pressed.

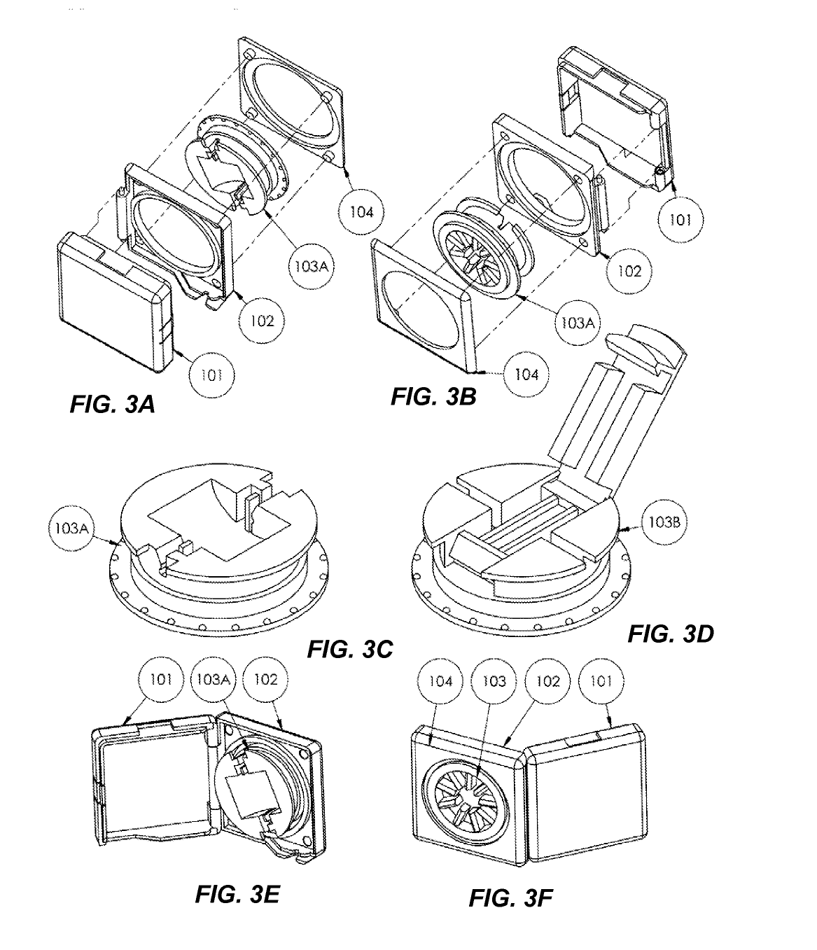

FIGS. 3A-8 depict an epicardial wire extraction system in accordance with one or more embodiments of the present invention. The system includes an epicardial wire extraction device and a disposable or replaceable cartridge.

Cartridge

The cartridge is depicted by way of example in FIGS. 3A-3F. The cartridge (100) consists of a small, disposable, plastic container in which the TEPW collects immediately after its extraction from the body. Its purpose is, firstly, for management of the extracted TEPW. The cartridge encloses the wire and eliminates the need for superfluous manoeuvres that would otherwise be required to control a loose and dangling TEPW. Secondly, the cartridge acts as a barrier between the contaminated TEPW and the hospital environment. Isolating the TEPW could potentially reduce the risk of spreading any pathogens that may lie on the wire's surface.

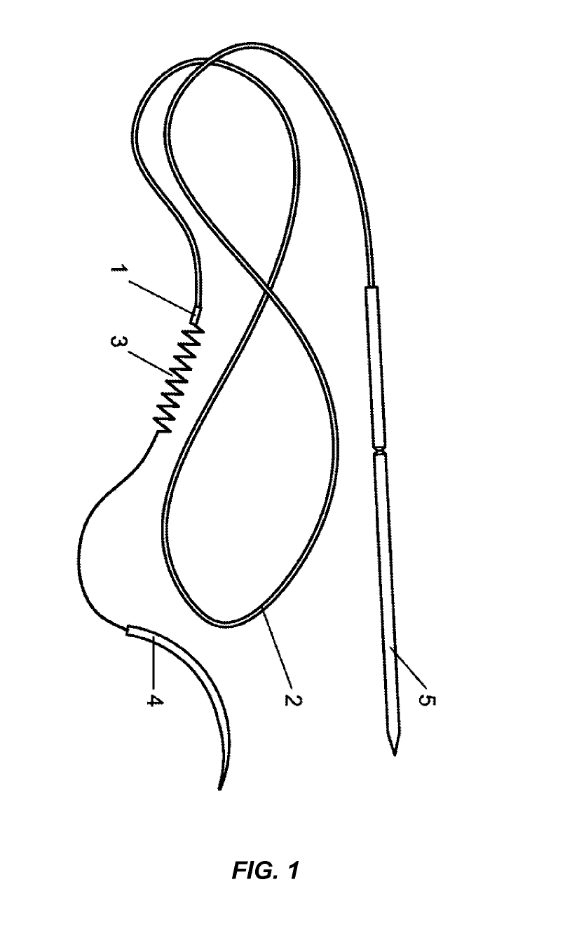

When beginning the TEPW extraction procedure, a new and unused cartridge is opened by pulling the lid (101) apart from the cartridge body (102). Doing this reveals the interior portion of the spool (103). Two interchangeable variations of the spool exist to accommodate two different TEPW external end conditions. The first (103A) is designed to accept a TEPW with a metal tip, which has been left over after the breakaway needle (5) shown in FIG. 1 is snapped off. In this case, the metal tip is aligned with the groove on the spool's face, centered along the diameter of the spool, and pushed downward until the metal piece snaps securely into place along the groove.

The second spool variation (103B) is designed to accept a TEPW from which the entire metal tip has be removed. In this case, the end of the insulated wire is placed inside the small groove that crosses the surface of the spool. The folding clamp, oriented perpendicular to this groove, is snapped into place on top of the wire's end, securing it into place.

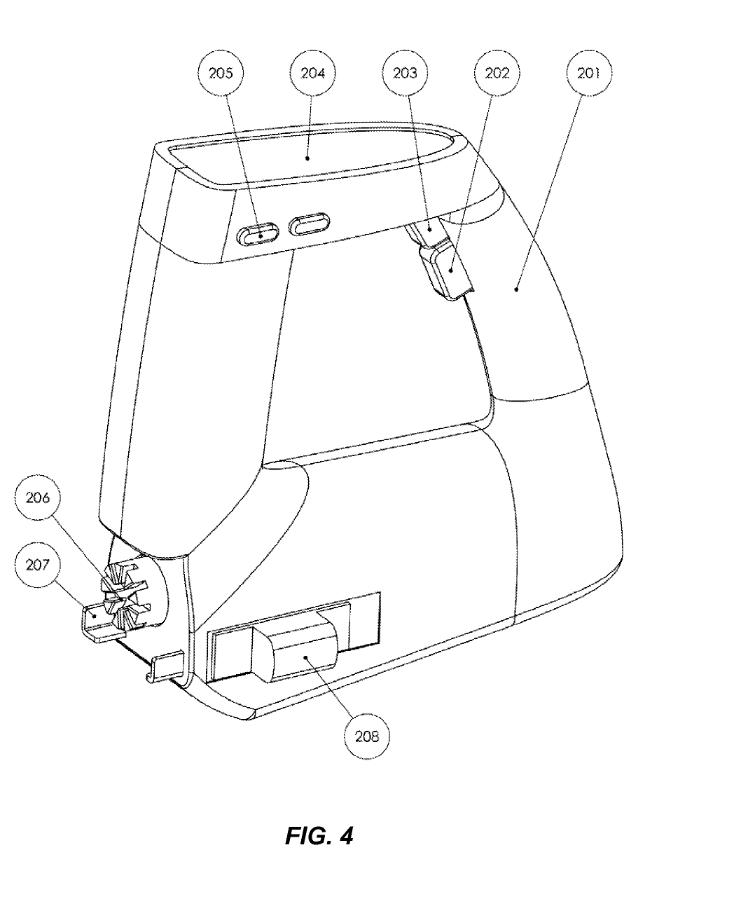

In both variations, the spool component is loosely held between the cartridge's main body (102) and the cartridge cap (104). The cartridge's body and cap are designed to allow the spool to shift laterally in any direction but restrict its displacement along the central axis of the spool. This freedom of lateral motion allows the load applied to the spool by the wire to be transferred directly to the device without passing through the cartridge body (102). The reason for this feature is elaborated upon below. The portion of the spool that faces outwards has a coupling which meshes into the device's matching coupling (206) as illustrated in FIG. 4. The spool's flange has spherical protrusions along its perimeter to reduce friction against the cartridge's body when the spool is rotating.

Once the external end of the TEPW is fixed to the spool in one of the two methods previously explained, the wire is guided into the curved notch (clearly seen in FIG. 3E) on the side of the cartridge's body and the lid is snapped shut. With the lid closed, the wire still moves freely within the notch.

Handle

In the embodiment depicted by way of example in FIG. 4, the device handle (201) is at a slight forward angle off the vertical to allow the operator to comfortably hold the device on the patient with their arm at 90 degrees which maximises control and stability. In one embodiment, a bottom surface of the housing is shaped to rest against an abdomen of a patient. For example, the bottom surface may be substantially flat or may be curved with a curved concave underside to fit the patient's anatomy. Two buttons are located on the interior surface of the handle. When pressed, the top button (203) triggers the release of the cartridge. The button is pressed at the end of the procedure when the TEPW is completely wound inside the cartridge, or if the operator deems that the procedure should be aborted during wire retraction. In another embodiment, the device has one stop/start trigger button (202) on the bottom surface of the handle and one cartridge release button (203) on the top of the handle where the thumb would lie.

The bottom button (202) is the start/stop trigger. Once the loaded cartridge is attached to the device, holding the bottom button will initiate the extraction sequence. As explained in detail below, the motor will start up and supply the necessary torque and rotational speed to the spool. The rotational speed is completely controlled by the device and is based on the stage of the wire extraction and the tension force measured by the device. At any point, the bottom button can be released to pause the TEPW extraction without releasing the cartridge.

In the case that the tension force applied to the wire approaches the predetermined maximum threshold, the handle will vibrate to provide a tactile warning to the operator. This allows the operator to focus their attention on the patient without having to constantly look at the device's screen for feedback.

Display Screen

In the embodiment illustrated by way of example in FIG. 4, the topmost surface of the device features a built-in display screen (204) which can display the following information: tension being applied on the TEPW, operational status of the device, battery charge, warnings for surpassing preset tension thresholds, and warnings for device misuses. Buttons (205) on either side of the screen allow the user to customize the display and input operational parameters such as tension thresholds and the speed of TEPW extraction. Alternatively, the display screen (204) may be a touch-screen adapted to display information and to receive user input directly on the screen. A touch-screen version need not have the physical buttons (205). Optionally, the device may include additional buttons or user interface elements to program or set thresholds or to perform diagnostics or calibrations, etc.

Cartridge Holder and Release Mechanism

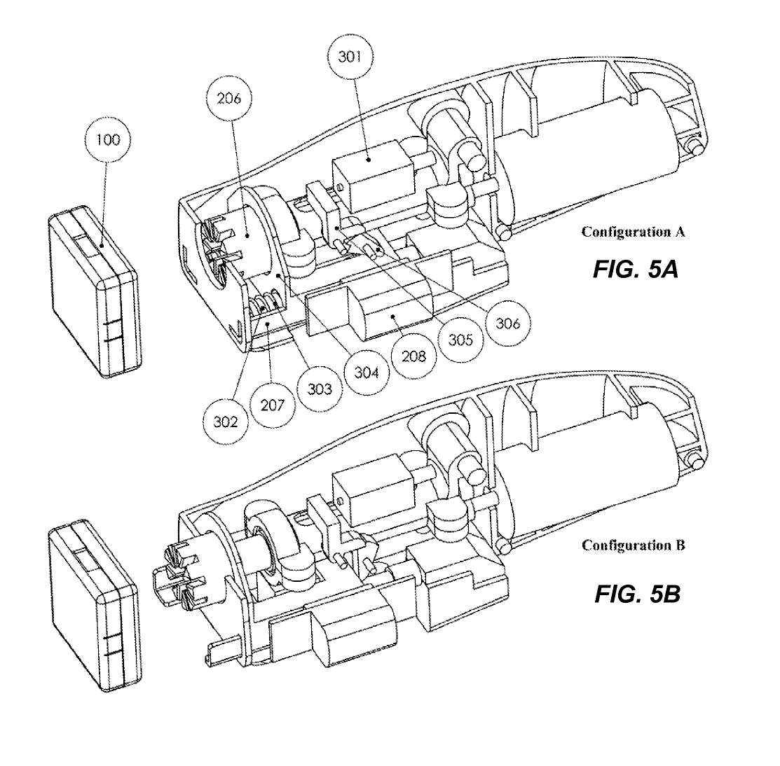

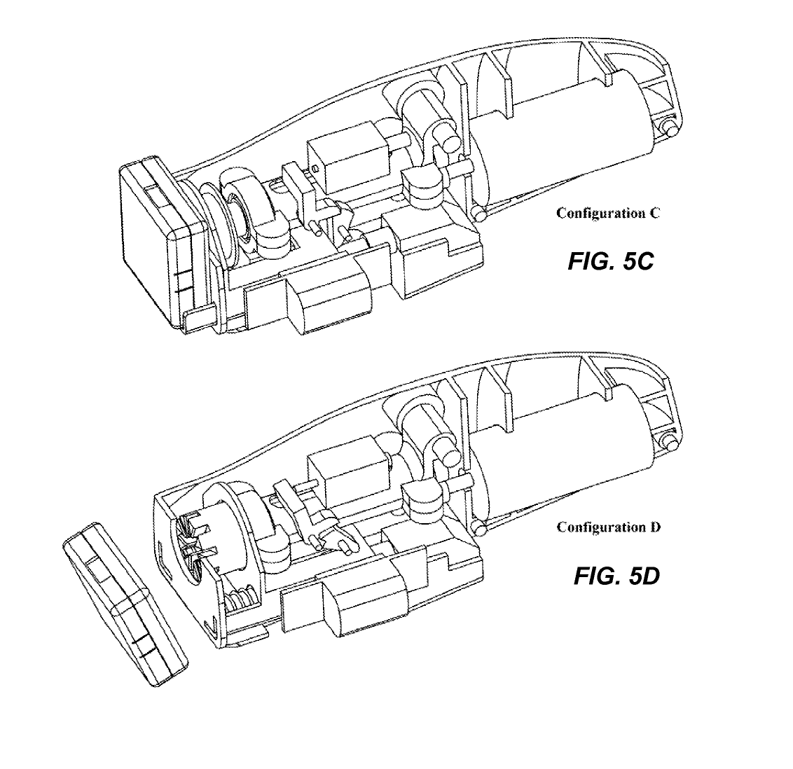

The cartridge holder and release mechanism are depicted by way of example in FIGS. 5A-5D.

When not in use, the device is in its rest state (Configuration A) during which the coupling (206) and the cartridge holder (207) are in a retracted position inside the device's frame.

Once the external end of the TEPW has been securely fastened inside the cartridge (100) (as explained above), the cartridge can be mounted to the front of the device. To do this, the cartridge holder and coupling are brought out by pushing forward on the slider (208), located on the left side of the device. The slider is attached to the cartridge holder frame (304), therefore pushing it causes the cartridge holder frame to move forward along two guide rods (302) positioned symmetrically on either side. As the cartridge holder frame advances, springs (303) wrapped around the guide rods are compressed. When the cartridge holder frame reaches its foremost position, the spring-loaded sear (306) locks into place behind the cartridge holder frame, preventing it from springing back to its rest position. At this point (Configuration B), the mechanism is cocked and the cartridge holder (which is really just the externally visible extension of the cartridge holder frame) protrudes out the front of the device. In addition, the spring-loaded coupling, having followed the displacement of cartridge holder frame, emerges from the device.

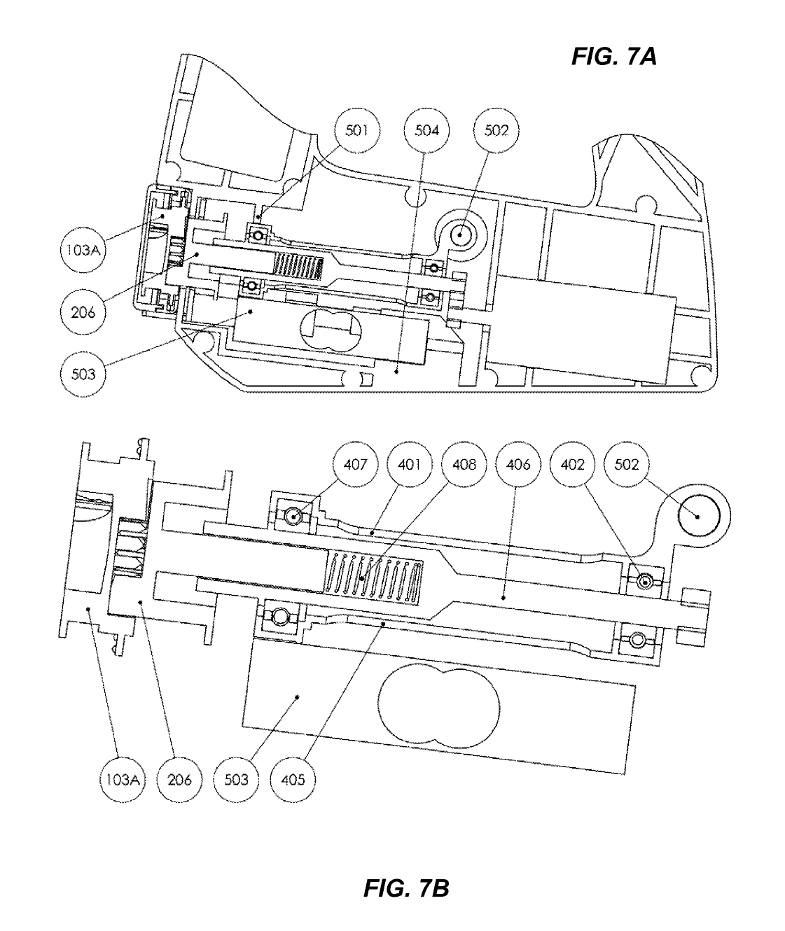

The cartridge is then slid into position (Configuration C) between the two arms of the cartridge holder and below the lip formed by the device's frame (shown in FIG. 7A).

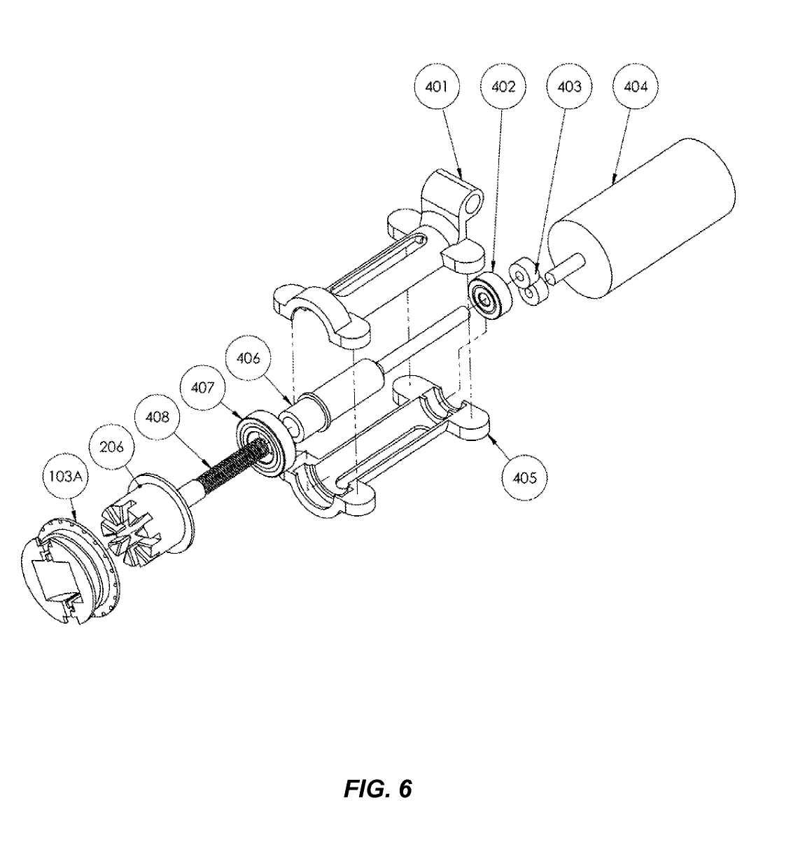

In the case that the device and spool couplings do not immediately align, the device's coupling is forced back into the device to make room for the cartridge. However, as soon as the device's coupling begins to rotate and align itself with the cartridge's coupling, they are forced to mesh together by a spring (408) as illustrated in FIG. 6 or FIGS. 7A and 7B) located behind the device's coupling.

When the eject button is pressed, the measured tension exceeds the maximum threshold, or a safety feature activates, an electrical signal is sent to the solenoid actuator (301) causing its shaft to strike the trigger (305) (Configuration D). The trigger releases the sear, and the cartridge holder frame is quickly forced back by the compressed springs around the guide rods. As a result, the cartridge holder and coupling are retracted back into the device, releasing the cartridge. In another embodiment, the device may include an electromagnet to latch the mechanism instead of the trigger and sear. When charged the electromagnet holds the cartridge holder frame in its cocked position and releases the mechanism when discharged. This provides an additional safety feature in the case that power is unexpectedly cut preventing the solenoid from striking the trigger.

Powertrain

An exemplary powertrain of the device is depicted in FIG. 6 although it will be appreciated that other powertrain designs may be utilized to achieve a similar result.

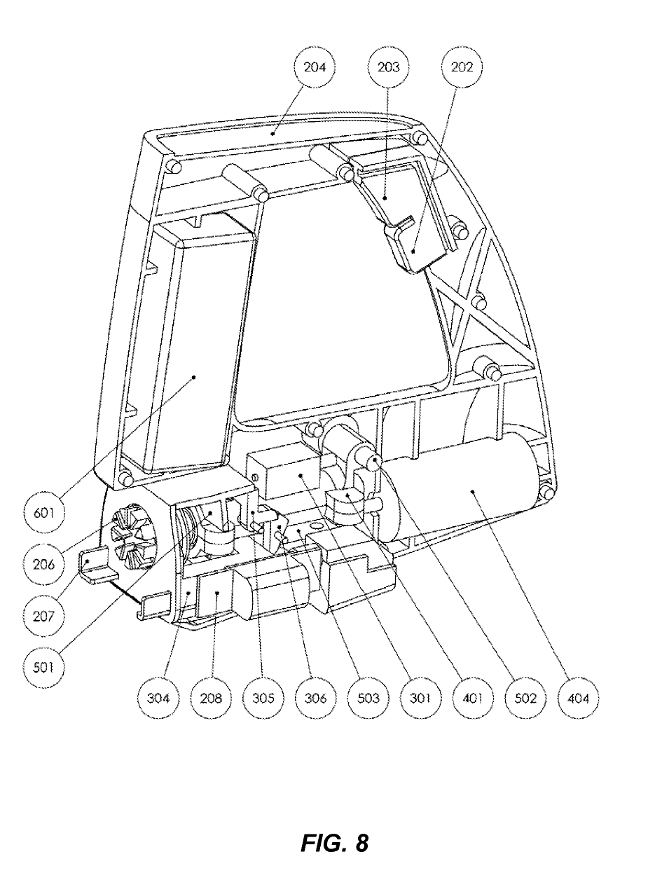

The angular velocity and torque required to wind the TEPW around the cartridge's spool (103) is supplied by a DC gear motor (404). The gear motor is powered by an onboard battery (601) as illustrated in FIG. 8 and includes a built-in reduction gear train which reduces the output angular velocity of the motor, while increasing the output torque. The battery (601) may be a rechargeable battery, e.g. lead—acid, nickel cadmium (NiCd), nickel metal hydride (NiMH), lithium ion (Li-ion), or lithium ion polymer (Li-ion polymer) or any other suitable type. The rechargeable battery could optionally be recharged by induction by placing it on a charging stand. In other embodiments, the battery may be a non-rechargeable battery or a plurality of batteries. In yet other embodiments, the device may have an electrical power cord for connecting to an external electrical power source, e.g. an electrical wall outlet. As illustrated in FIG. 6, the gear motor (404) transfers the torque through a pinion gear (403) on its output shaft to an identical gear on the drive shaft. The drive shaft (406) is mounted to two ball bearings (402, 407) and supported between the top drive shaft casing (401) and the bottom drive shaft casing (405). At the end closest to the cartridge, the drive shaft has a keyed cylindrical sleeve into which a spring (408) and the coupling's shaft (206) are inserted. The coupling is free to slide within the sleeve, but the key prevents the coupling from rotating with respect to the drive shaft. In this way, the rotational speed and torque of the drive shaft is transmitted directly to the coupling and then to the spool. The desired maximum rotational speed and torque have been calculated to be approximately 120 rpm and 23 oz-in, respectively.

Tension Measurement

In the embodiment illustrated by way of example in FIG. 7A and FIG. 7B, the tension measurement mechanism is incorporated into the powertrain since the tension force on the TEPW is applied directly to the spool (103) which is, in turn, connected to the device's drive shaft (406) through the coupling (206). The drive shaft casing (401, 405), which contains the drive shaft and bearings (402, 407) is held in place inside the frame of the device by a pin (502). This connection forms a hinge joint which allows the casing to rotate freely about the pin's axis. However, the rotation of the drive shaft casing is restricted from the top by a segment of the device's frame (at point 501) and from the bottom by the load cell (503) or any other suitable force sensor. It is important to note that although the rotation of the drive shaft casing about the pin is completely restricted by the frame and the load cell, the casing is not in any way attached to either. In fact, the whole powertrain assembly (excluding the gear motor) is not fixed at any point along its length except at the pin. As a result, when a tension load is applied to the spool by the TEPW during extraction, the load is predominantly supported by the front end of the load cell. With its back end firmly fixed to the device frame (at point 504), the load cell is able to convert the load applied to its front end by the drive shaft casing into an electrical signal, the strength of which is directly proportional to the tension load applied to the spool. The signal strength is converted to a value representative of the force and subsequently compared to the values associated with the predetermined thresholds. In other embodiments, the load cell may be replaced by any suitable force sensor or transducer, i.e. any load-sensing component such as a force-sensing resistor, strain gauge, etc.

Safety Features

In order to reduce the risk of injury to the patient, several safety features have been implemented into the device. As previously explained, in the case that the tension being measured by the load cell approaches the preset maximum threshold, the handle will vibrate, a visual warning will be displayed on the screen, and an audible alarm will alert the operator. If the tension reaches the maximum threshold, the gear motor will immediately stop and the cartridge holder will retract to release the cartridge from the device. A gyroscope and accelerometer continuously monitor the angle and acceleration of the device. In the case that the device is tilted to an extreme angle that compromises its proper operation, or if the device is dropped, the cartridge will be released immediately. In addition, a proximity sensor located on the bottom surface of the device will trigger the release of the cartridge if the device loses contact with the patient during TEPW extraction. Releasing the cartridge eliminates any tension on the wire and allows the cartridge to move independently of the device. This protects the patient from being injured by excessive forces that may accidently be applied to the TEPW. This device is therefore designed to minimize or at least greatly reduce the possibility of myocardial bleeding or cardiac tamponade.

In the case that the operator deems it appropriate to abort the extraction procedure, the eject button on the handle will also immediately release the cartridge. Lastly, if the battery does not have adequate charge to confidently complete an extraction procedure, the device will block the operator from starting the device.

The primary purpose of the tension-limiting temporary epicardial pacing wire extraction device is to provide a safer alternative to the manual removal of epicardial pacing wires. With this in mind, the device was designed to eliminate the subjectivity inherent in the current extraction method by quantifying and continuously monitoring the tension applied to the wire, and aborting the procedure in the event of excessive force. The device may include a processor (e.g. a microprocessor, microcontroller, control circuit, Programmable Logic Controller (PLC) or other control logic in the device) for receiving, monitoring, and comparing signals or readings against respective thresholds and for generating control signals to actuate the DC gear motor and solenoid actuator, generate displayable information on the display screen or to output other visual, audible and/or tactile alerts.

To recap, and as illustrated in FIGS. 3A-8, the device has a housing that houses a motor (404) powered by a battery (601) disposed within the housing. The housing may optionally have a substantially flat bottom surface or a contoured surface that is shaped to rest against the abdomen of a patient.

The device includes a handle (201) for holding the device with a single hand. The system includes a cartridge (100) containing a spool (103) driven by the motor (404) via the powertrain of FIG. 6 for extracting an epicardial pacing wire attached to the heart of a patient. The device includes a start/stop button (202) to operate the motor to drive the spool. The buttons (202, 203) are ergonomically disposed on the handle to be easily pressed by a forefinger while holding the handle of the device. The device includes a cartridge-release mechanism (of FIGS. 5A-5D) that selectively releases the cartridge from the housing. As depicted in FIG. 8, the handle optionally has a cartridge-release button (203) above the start/stop button (202). As noted above, in another embodiment, the stop/start trigger button (202) may be on the bottom surface of the handle and the cartridge release button (203) may be on the top of the handle. A load cell (503) acting as a force sensor generates a signal representative of the tension in the wire. As depicted in FIG. 8, the device further includes an optional LCD or LED display screen (204) for displaying information based on the signal generated by the force sensor (load cell).

The device is tension-limiting in that the excessive tension exerted on the wire may be mediated manually (i.e. in response to user input or control) or automatically (by sensing a tension overload condition or any other condition of improper usage). In the illustrated embodiment, the cartridge-release mechanism may be actuated by pressing a cartridge-release button. The cartridge-release mechanism may also be actuated in response to sensing a tension in the wire that exceeds a predetermined threshold. The cartridge-release mechanism may also be actuated in response to sensing a tilt and/or acceleration of the device that exceed predetermined tilt and/or acceleration thresholds. The cartridge-release mechanism may also be actuated in response to sensing that the device is no longer proximate to a body of a patient. It will be appreciated that the tension-limiting device may limit tension by any combination of the above features. In addition to the load cell (force sensor) and the cartridge-release button (“eject button”), the device may include a gyroscope for sensing an angle of the device and/or an accelerometer for sensing an acceleration of the device and/or a proximity sensor located on the bottom surface of the device for sensing a proximity of a body of the patient. The signals from these sensors are received by and processed by a processor. The processor compares the tilt angle to a predetermined angle threshold, compares the acceleration to a predetermined acceleration threshold, the proximity to a predetermined proximity (or distance) threshold to determine whether the device is operating within the correct ranges. As another safety feature, the device may be configured to not start up if a minimum battery charge is not present to fully complete the procedure.

If any of the thresholds are exceeded, the processor automatically causes the motor to stop and the cartridge-release mechanism to release the cartridge, i.e. in response to sensing a tension overload condition or sensing that the angle exceeds a predetermined angle threshold or that the acceleration exceeds a predetermined acceleration threshold or that the device is no longer proximate to the body of the patient.

In the illustrated embodiment, the cartridge-release mechanism comprises a solenoid actuator that receives a control signal from the processor. When actuated, a shaft of the solenoid strikes a trigger to release a sear to enable a cartridge holder frame to be forced back by compressed springs into the housing to thereby release the cartridge. In another embodiment, the cartridge-release mechanism comprises a electromagnet or any other suitable latching mechanism capable of holding the cartridge holder frame which can be actuated automatically or manually to retract the cartridge holder. The device may further include a memory coupled to the processor for storing tension data or for storing user-configurable thresholds, settings or limits. For example, in one embodiment, the operating limits or thresholds may be programmable or reconfigurable by the attending cardiologist.

Any ratios or proportions of the components of the device shown in the figures are specific to the illustrated embodiments. As will be appreciated by those skilled in the art of mechanical design, these dimensions, ratios or proportions may be varied to achieve different size and/or performance requirements.

This new technology has been described in terms of specific implementations and configurations which are intended to be exemplary only. Persons of ordinary skill in the art will appreciate that many obvious variations, refinements and modifications may be made without departing from the inventive concepts presented in this application. The scope of the exclusive right sought by the Applicant(s) is therefore intended to be limited solely by the appended claims.

What is claimed is:

1. An epicardial pacing wire extraction system comprising:
an epicardial pacing wire extraction device comprising:
a housing that houses a motor;
a handle for holding the device;
a start/stop button to operate the motor; and
a cartridge-release mechanism; and
a cartridge containing a spool driven by the motor for extracting an epicardial pacing wire from a patient and being selectively releasable from the device.

2. The system as claimed in claim 1 wherein the cartridge-release mechanism is actuated by pressing a cartridge-release button.

3. The system as claimed in claim 1 wherein the cartridge-release mechanism is actuated in response to sensing a tension in the wire that exceeds a predetermined threshold.

4. The system as claimed in claim 1 wherein the cartridge-release mechanism is actuated in response to sensing a tilt and/or acceleration of the device that exceed predetermined tilt and/or acceleration thresholds.

5. The system as claimed in claim 1 wherein the cartridge-release mechanism is actuated in response to sensing that the device is no longer proximate to a body of a patient.

6. The system as claimed in claim 1 wherein the device further comprises a force sensor for sensing a force applied by the device to the wire.

7. The system as claimed in claim 6 wherein the device further comprises a display screen for displaying information based on a signal generated by the force sensor.

8. The system as claimed in claim 1 wherein the device further comprises a processor for determining if a tension measured by a force sensor reaches a maximum threshold and for causing the motor to stop and a cartridge holder to retract to release the cartridge.

9. The system as claimed in claim 1 wherein the device further comprises:
a gyroscope for sensing an angle of the device;
an accelerometer for sensing an acceleration of the device; and
a processor for causing the cartridge-release mechanism to release the cartridge in response to sensing that the angle exceeds a predetermined angle threshold or the acceleration exceeds a predetermined acceleration threshold.

10. The system as claimed in claim 1 wherein the device further comprises:
a proximity sensor located on the bottom surface of the device for sensing a proximity of a body of the patient;
a processor to cause the cartridge-release mechanism to release the cartridge in response to sensing that the device is no longer proximate to the body of the patient.

11. The system as claimed in claim 1 wherein the device further comprises:
a force sensor for measuring a tension in the wire;
a gyroscope for sensing an angle of the device;
an accelerometer for sensing an acceleration of the device;
a proximity sensor for sensing a proximity of the device to a body of the patient; and
a processor for causing the cartridge-release mechanism to release the cartridge in response to any one of: (i) sensing that the tension exceeds a predetermined tension threshold, (ii) sensing that the angle exceeds a predetermined angle threshold, (iii) sensing that the acceleration exceeds a predetermined acceleration threshold and (iv) that the device is no longer proximate to the body of the patient.

12. An epicardial pacing wire extraction device comprising:
a housing that houses a motor;
a handle for holding the device;
a start/stop button to operate the motor; and
a cartridge-release mechanism.

13. The device as claimed in claim 12 wherein the cartridge-release mechanism is actuated by pressing a cartridge-release button.

14. The device as claimed in claim 12 wherein the cartridge-release mechanism is actuated in response to sensing a tension in the wire that exceeds a predetermined threshold.

15. The device as claimed in claim 12 wherein the cartridge-release mechanism is actuated in response to sensing a tilt and/or acceleration of the device that exceed predetermined tilt and/or acceleration thresholds.

16. The device as claimed in claim 12 wherein the cartridge-release mechanism is actuated in response to sensing that the device is no longer proximate to a body of a patient.

17. The device as claimed in claim 12 wherein the display screen also displays one or more of operational status, battery charge, a warning for surpassing a preset tension threshold, and a warning for misusing the device.

18. A cartridge for use with an epicardial pacing wire extraction device to extract an epicardial pacing wire, the cartridge comprising:
a casing for containing the epicardial pacing wire during and after extraction; and
a spool held inside the casing, the spool attaching to an external end of the epicardial pacing wire, the spool having an external portion driven by a torque-transmitting coupling of the device.

19. The cartridge as claimed in claim 18 wherein the casing has a hinged lid providing access to the spool and wherein, upon closing, the lid is locked shut to prevent reuse of the cartridge.

20. The cartridge as claimed in claim 18 wherein the casing allows the spool to rotate and shift laterally over a limited distance with respect to the casing but restricts translational motion of the spool along an axis of the spool, thereby transferring a load imposed on the spool by the wire to the device for measurement.
